(19) United States
(12) Patent Application Publication
(10) Pub. No.: US 2004/0146943 A1
(43) Pub. Date: Jul. 29, 2004

(54) Novel composition and use as matrix in MALDI-TOF-MS

(76) Inventor: Martin C. Wilkes, Rockton, IL (US)

Correspondence Address: Pierce Biotechnology, Inc., Attn: Thomas E. Currier, P. O. Box 117, Rockford, IL, 61105, US

(21) Appl. No.: 10/760,994

(22) Filed: Jan. 20, 2004

Related U.S. Application Data
(60) Provisional application No. 60/441,214, filed on Jan. 21, 2003.

(51) Int. Cl. G01N 33/53

(52) U.S. Cl. 435/7.1; 562/465

(57) ABSTRACT

The novel compound 3-(4-Hydroxy-2-trifluoromethyl-phenyl)-acrylic acid and its use as a matrix in matrix-assisted laser desorption ionization-time of flight-mass spectrometry are disclosed.

### RELATED APPLICATION

This application claims priority of U.S. Provisional Application Serial No. 60/441,214, filed Jan. 21, 2003.

### FIELD OF INVENTION

The present invention relates to the method commonly referred to as MALDI-TOF-MS which is an acronym for matrix-assisted laser desorption ionization-time of flight-mass spectrometry and to a new compound useful as the matrix in MALDI-TOF-MS.

### BACKGROUND OF INVENTION

MALDI-TOF-MS is finding increased use in the determination of the mass of large, non-volatile biomolecular analytes. In the method, laser pulses are focussed on a sample plate containing the analytes embedded in a matrix. The matrix absorbs most of the energy of the laser and, in turn, through proton transfer, ionizes and vaporizes the analyte molecules.

The gaseous ions are then transferred into a time-of-flight mass spectrometer. Therein, the molecules are accelerated through a strong electric field and then, based on mass to charge ratios, separated from each other by passage through a field-free region. The molecules are then detected by collision with a detector that generates a signal as each set of ions of a particular mass to charge ratio strikes the detector. The mass of the molecule correlates to the time it takes for the molecule to travel from the sample plate to the detector and can thus be determined by time-of-flight mass spectrometric analysis. Additional discussion related to MALDI-TOF-MS can be found in the following items, the entire disclosures of which are herein incorporated by reference: U.S. Pat. No. 6,104,028; “The Scientist” 13[12]:18, Jun. 7, 1999; and “Biophotanics International”, June 2001, 42-47.

In MALDI-TOF-MS, the selection of the matrix is important. The matrix is generally a small organic compound that can absorb intense laser energy thus preventing decomposition of the analyte and yet gently transfer energy to it and promote ionization. See Zenobi and Knochenmuss, “Mass Spec Rev 17” (1998) 337, which is entirely incorporated herein by reference.

### SUMMARY OF THE INVENTION

In accordance with the present invention, there is provided a new compound which can be used as a matrix in MALDI-TOF-MS as conventionally practiced. The novel material has the empirical formula C₁₀H₇F₃O₃ and is chemically named 3-(4-Hydroxy-2-trifluoromethyl-phenyl)-acrylic acid, hereinafter referred to as “HTFPA”. The compound, HTFPA, can be represented by the following structure:

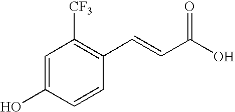

3-(4-Hydroxy-2-trifluoromethyl-phenyl)-acrylic acid

C₁₀H₇F₃O₃

Exact Mass: 232.03

Mol. Wt.: 232.16

C, 51.74; H, 3.04; F, 24.55; O, 20.67

### DESCRIPTION OF INVENTION, INCLUDING EXAMPLES

The use of HTFPA in MALDI-TOF-MS is accompanied by the following attributes:

1) A broad cocrystallization range (1-138K) for preparations with glycoproteins,proteins and peptides

2) Matching of chromophore matrix oscillator strength to nitrogen laser wavelength.

(Used in major market MALDI mass spec instruments, eg. (PE Voyager/Micromassinstruments). Excellent matrix volatility in laser plume.

3) Water and solvent solubility.

3) Ability to absorb more laser power to better protect the protein during the ionization process, a feature especially useful for glycoproteins such as fetuin)

Particular utility for MALDI-TOF-MS applications using the HTFPA matrix are

considered to be:

1) Applications for proteomics:

A. Characterizing unknown novel proteins/peptides.

B. Characterizing enzyme cleaved protein fragments.

C. Disease diagnosis where a metabolite is produced.

2) Potential for microcrystal growth in X-Ray/e-beam crystal structures of proteins.

3) Potential for DNA/RNA mass spectrometry.

HTFPA Preparation

HTFPA can be prepared as follows:

First, the precursor, 4-Bromo-3-trifluoromethyl-phenol (BTFP) is prepared by adding 50 g of commercially available (Acros) 3-trifluoromethyl-phenol and 25 mL chloroform to a 250 mL morton flash with stirbar and Friedrichs condenser, ice cooled in a Dewar dish. 55 g Bromine is added dropwise allowing time for HBr evolution. Excess HBr is spurged out under nitrogen gas and a second treatment with 5 g bromine is accomplished. On stripping 50.4 g crude BTFP product is obtained. 15 g pure BTFP isomer, m.p. 44-46 C., is then obtained by elution with 50% hexanes/50% dichloromethane from a 1-liter, 70-230 mesh silica gel column ( Aldrich), dry packed and conditioned with 75% hexanes/25% dichloromethane.

From this precusor, HTFPA is then prepared in the following manner:

1) 7.78 g BTFP (prepared as described above), 12.25 g triethylamine, 0.196 g triorthotoluylphosphine (Aldrich), 2.90 g acrylic acid, 72.3 mg palladium diacetate (Acros) and 8 mL DMF are added to a 500 mL morton flask with stirbar, thermometer, and Friedrichs condenser, under nitrogen.

2) The solution is heated to 105 C. external and 90 C. internal temperature.

3) The solution is sampled at intervals at NMR testing (CDC13/ldrop d6DMSO). Samples are extracted with dichloromethane/10% HCl to remove TEA. (If the catalytic cycle stops (i.e., indicated by a black Pd ppt) then restart with additional palladium acetate and repeat).

4) When the starting material is gone, the reaction solution is cooled and extracted with 3×200 mL ether and the ether layer washed with 2×200 mL 10% HCl.

5) The organic layer is stripped and redissolved in dichloromethane and poured through a 50 mL pad of silica gel (70-230 mesh from Aldrich). The product is eluted off with ethyl acetate (removes any polyacrylate present). On stripping, 50.4 g crude product is obtained.

6) A 600 mL silica gel column is dry packed and conditioned with 10/100/890 Acetic acid/ethyl acetate/hexanes which is also used to elute off phenol impurities. This is followed by elution with 25/225/750 Acetic acid/ethyl acetate/hexanes which also removes residual phenols and finally 33/300/667 acetic acid/ethyl acetate/heanes to elute off 0.5 g of crude crystals which is recrystallized from 50 mL hot 1/6 ethyl ether/hexanes.

7) The recrystallized product, substantially pure HTFPA, has a melting point of 167-178 C. and proton/C-13 NMR Use of HTFPA as a matrix in MALDI-TOF-MS is illustrated below.

Matrix Preparation:

Solutions of HTFPA are prepared at a concentration of 10 mg/ml in acetonitrile:water (with 0.1% trifluoroacetic acid) at 50:50 v/v.

Sample Preparation:

Samples for MALDI-TOF MS analysis, containing analytes embedded in HTFPA are prepared by mixing 2 μL of the aqueous protein/peptide solutions identified below with 2 μL of matrix solution. 1 μL of the mixed solution is deposited on a target plate and dried under room temperature before analysis. The protein/peptide solutions are as follows: Angiotensin I at 10 pmol/μL; Insulin at 1 mg/mL; Myoglobin at 1 mg/mL; Fetuin at 1 mg/mL; and BSA at 1 mg/mL.

MALDI-TOF-MS:

A Micromass TOFSPEC-2E is employed for the analyses. For linear mode experiments, the sampling rate is 250 MHz. In the reflectron mode, the sampling rate is 2 GHz. Each spectrum consists of 16 laser shots. Ten to fifteen spectra from each acquisition were combined for comparison purposes.

Experiment Results:

For the analysis of Angiotensin I, the matrix used in the study yielded peak at m/z 1297.

For the analysis of the mixture of Insulin and Myoglobin, both matrices produced pseudo-molecular ions of insulin and myoglobin, For the analysis of BSA, the matrix produced the pseudo molecular ion of BSA at m/z 66341, a dimer at m/z 132861 and doubly charged peak at m/1 33216. For the analysis of Fetuin, the matrix produced the pseudo-molecular ions of fetuin at m/z 47000, a dimer peak and a doubly charged peak as well.

A preferred embodiment of this invention is described herein, including the best mode known to the inventor for carrying out the invention. Variations may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. The compound 3-(4-Hydroxy-2-trifluoromethyl-phenyl)-acrylic acid.

2. In a process for the determination of the mass of a biomolecular analyte by matrix-assisted laser desorption ionization-time of flight-mass spectrometry comprising vaporizing the analyte by focussing laser pulses on a matrix containing the analyte and then tranferring the vaporized analyte into a time of flight-mass spectrometer, the improvement wherein the matrix is the compound 3-(4-Hydroxy-2-trifluoromethyl-phenyl)-acrylic acid.
